(19) United States
(12) Patent Application Publication
(10) Pub. No.: US 2010/0120073 A1
(43) Pub. Date: May 13, 2010

(54) METHODS AND DEVICES FOR DETECTING ORGANISMS CAUSING URINARY TRACT INFECTIONS

(75) Inventors: Chad A. Hanson, Hugo, MN (US); Geoff H. Gorres, Amery, WI (US)

Correspondence Address: SHERRILL LAW OFFICES, 4756 BANNING AVE, SUITE 212, WHITE BEAR LAKE, MN, 55110-3205, US

(73) Assignee: SUPERIOR MEDICAL LLC, Hugo, MN (US)

(21) Appl. No.: 12/598,234

(22) PCT Filed: May 7, 2008
(86) PCT No.: PCT/US08/62836

§ 371 Date: Oct. 30, 2009

Related U.S. Application Data
(60) Provisional application No. 60/916,722, filed on May 8, 2007.

(51) Int. Cl. G01N 33/569; C12M 1/34; G01N 33/53

(52) U.S. Cl. 435/7.33; 435/287.7; 435/287.2; 435/7.32; 435/7.37

(57) ABSTRACT

The invention provides for devices and methods for detecting an organism causing a urinary tract infection. In a preferred embodiment, detecting the organism causing a urinary tract infection follows a preliminary indication of a urinary tract infection by a urinary monitoring device to monitor for the presence or absence of markers indicative of a urinary tract infection. The invention also provides for methods of using such devices.

This application claims the benefit of United States Provisional Application No. 60/916,722, filed May 8, 2007.

### TECHNICAL FIELD

This invention relates to medical devices and methods, and more particularly to methods, devices, and systems for identifying organisms causing urinary tract infections and resistance factors and the like related thereto.

### BACKGROUND

Urinary tract infections (hereafter “UTI” or “UTIs”) are a major cause of morbidity and mortality in healthcare, especially in hospitalized or otherwise debilitated patients. The risk of infection is substantially increased in patients having a urinary catheter. So much so that among the diagnosed UTIs, catheter-associated urinary tract infections (hereafter “CAUTI” or “CAUTIs”) are the most common nosocomial infections, accounting for more than 40% of all hospital-acquired infection. In addition, many afflicted patients that are further debilitated are unable to effectively communicate their symptoms. Unfortunately for those patients unable to express their symptoms, their infections may go unrecognized until the infection enters advanced stages such as life-threatening sepsis.

CAUTIs are the second most common cause of nosocomial sepsis after pneumonia. More than 750,000 patients in the United States develop severe sepsis each year, which is characterized by acute organ system dysfunction. The mortality rate from severe sepsis, at 28.6%, kills 215,000 Americans annually at an estimated cost of about $16.7 billion. That translates into nearly 600 patients dying each day—which means that as many patients in the United States die from severe sepsis each day as die from acute myocardial infarction.

Due to the life threatening nature of CAUTIs, it is desirable to discover and treat a CAUTI as early as possible. Urine dipsticks or strips are available (e.g., Multistix®, Bayer, Leverkusen, Germany; Chemistrip®, Roche Diagnostics, Indianapolis, Ind.; Multistix® 10 S G, Miles Laboratories, Inc., Elkhart, Ind.; and Combur-Test®, Boehringer Mannheim Corp., Indianapolis, Ind.). Dipsticks require an index of suspicion, are labor-intensive for nursing staff, and usually require incident-specific physician orders. As a result, urine specimens are commonly sent to a hospital or central laboratory only when someone observes indicators like cloudiness, color change, or visual signs of blood in the urine.

Accepted practices can result in a significant lapse of time before a UTI is clinically diagnosed and treatment initiated. Ultimately, the cost of these infections in both dollars and human life is substantial. U.S. patent application Ser. No. 11/318,769 filed on Dec. 27, 2005, and entitled System and Device for Detecting Markers Associated with a Urinary Tract Infection (hereafter “UMD inventions”) discloses systems, devices, and methods for monitoring a patient for a UTI and providing an early indication of a UTI. The UMD inventions are especially useful for early detection of a CAUTI.

Clinical outcomes could be improved, however, by providing specific information regarding the causative organism of a UTI, including CAUTIs, earlier in the infection cycle. Such information may provide more timely, targeted, and cost-effective ways to combat UTIs and CAUTIs. The present disclosure provides devices, systems, and methods that allow for rapid identification of the causative organism of UTIs in inpatient or outpatient settings and in catheterized or non-catheterized patients. This timely information may allow for earlier, targeted, and specific therapies that save money, lives, and improve clinical outcomes for afflicted patients.

### SUMMARY OF THE INVENTION

In one aspect, the systems, devices, and methods described herein provide timely, sensitive, and specific information regarding the presence or absence of markers in the urine that are indicative of a UTI, including a CAUTI, as well as the causative organism of the infection, and bacterial resistance factors. In preferred embodiments, the systems, devices, and methods described in this patent will work in conjunction with the systems, devices, and methods of the UMD inventions. In this way, the invention may provide timely information to allow for earlier, targeted, and specific therapies that save money, lives, and improve clinical outcomes for afflicted patients. For example, a medical professional may detect a UTI earlier and diagnose the causative organism earlier allowing earlier treatment targeted at a specific organism.

The markers of the present inventions may be tested for in single or multiple fashion, and the assays may be run in parallel or in series (see Figures). In some instances the urine may be run directly on the reagent substrate, and in others it may require processing with reagents in order to better present the specific anticipated antigens. The assays described may also incorporate control elements and solutions in methods known in the art.

In one aspect, the invention provides for a device for indicating the causative organism of a urinary tract infection including a urine-sample receiver connected to a housing, the housing encasing a carrier, the carrier configured to receive urine entering the housing from the urine-sample receiver, wherein the carrier supplies the urine into a plurality of causative-organism-detection reservoirs, the reservoirs adapted to detect the presence of an urinary-tract-infection organism, and wherein the reservoirs are viewable by at least one result window, the result window capable of providing a visible indication of the presence or absence of the urinary-tract-infection organism in the urine.

In some embodiments, the plurality of causative-organism-detection reservoirs include chemicals capable of detecting organisms selected from the group consisting of Escherichia, Staphylococcus, Klebsiella, Enterococcus, Proteus, Morganella, Pseudomonas, and Candida.

In some embodiments, the urine receptacle is connected to a catheter. In some embodiments, at least one of the causative-organism-detection reservoirs acts as a control. The control can include a positive control and a negative control.

In some embodiments, the detection of the presence of an organism that can cause a UTI is a plus sign, letter, or number. In some embodiments that viewing window may display positive and negative results. In some embodiments the readout of the device is taken manually by viewing the result window wile in others an automatic reader or detector may be adapted to read and report a result. The automatic detectors or readers may be part of or connected to a computer.

In some embodiments, the plurality of causative-organism-detection reservoirs employ antibodies. At times, the devices described in this patent are disposable.

In some embodiments, the plurality of causative-organism-detection reservoirs includes at least three causative-organism-detection reservoirs, at least five causative-organism-detection reservoirs, at least ten causative-organism-detection reservoirs, or more than ten causative-organism-detection reservoirs.

In another aspect, the inventions include an article of manufacture including a plurality of causative-organism-detection reservoirs for indicating the causative organism in urinary tract infections, the reservoirs containing substances to detect the presence of a urinary-tract-infection organism with the use of PCR amplification, and optionally a PCR device configured or programmed to accept the causative-organism-detection reservoirs, run a PCR reaction, indicate the presence or absence of a urinary-tract-infection organisms in urine.

In some embodiments, the device adapted for PCR use has a plurality of causative-organism-detection reservoirs include chemicals capable of detecting organisms selected from the group consisting of Escherichia, Staphylococcus, Klebsiella, Enterococcus, Proteus, Morganella, Pseudomonas, and Candida. The plurality of sample collection materials can be configured to collect urine samples from a urinary monitoring device and adapted to transfer the urine samples to the plurality of causative-organism-detection reservoirs for indicating the causative organism in urinary tract infections.

In another aspect, the invention features a method for detecting causative organisms of a UTI including the following steps: obtaining a urine sample; depositing the urine sample in an immunoassay device for indicating the causative organism of a urinary tract infection, the immunoassay device including a urine-sample receptacle connected to a housing encasing an absorbent material for receiving the urine that feeds portions of the urine sample into a plurality of causative-organism-detection reservoirs, the causative-organism-detection reservoirs providing a result that is visible through at least one result window that correspond to the plurality of causative organism detection reservoirs.

In another aspect, the invention features a device for indicating the causative organism in urinary tract infections including a thermocycler configured to perform a polymerase chain reaction method on a urine sample to detect the presence of absence of a urinary-tract-infection organism in the urine sample. In some embodiments, the thermocycler is configured to detect the presence or absence of the urinary-tract-infection organisms selected from the group consisting of Escherichia, Staphylococcus, Klebsiella, Enterococcus, Proteus, Morganella, Pseudomonas, and Candida.

In another aspect, the invention features a test kit for determining the presence of a causative organism in a urine sample including an assay device, the assay device having a plurality of detection zones, wherein the presence of a causative organism of a urinary tract infection in a urine sample is indicated by accumulation of a labeled reagent in one or more of the detection zones, and optionally wherein the accumulation is visible through a window in the assay device. In some embodiments, the plurality of detection zones that include ELISA or PCR technology. In other embodiments, the assay device includes a carrier strip disposed within a hollow housing, the carrier strip having the detection zone, and wherein an assay result is revealed by specific binding of the labeled reagent within the detection zone. In some embodiments, the assay result displays a plus sign for a positive result and a negative sign for a negative result.

In another aspect, the inventions feature a device for monitoring urine including a presence of a causative organism in a urine sample including an assay device, the assay device configured for placement in-line in a urinary catheter system, wherein the urinary catheter system includes a urinary catheter, a collection tube, and a collection bag, wherein the assay device includes a urine entry port and a urine exit port, the assay device having a plurality of detection zones, wherein the presence of a UTI-causative organism in a urine sample is indicated by accumulation of a labeled reagent within one or more of the detection zones, wherein the accumulation is visible through a window in the assay device.

In some embodiments, the systems and devices of this patent can further comprise a transmitter for communicating the signal or results over a distance. By way of example, a signal can be communicated digitally or via a computer, and a signal also can be communicated together with other vital signs of a patient. In some instances, the system can be programmable.

It is to be understood that the various aspects of the embodiments described or envisioned herein may be mixed and matched to provide a greater variety of embodiments. Further the methods and devices of these inventions can work in conjunction with all types of catheters, including known Foley catheters, male external catheters, female incontinence catheters, anti-infection catheters, and other similar designs.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, books, and other references mentioned anywhere in this application are incorporated by reference in their entirety. And for patents and patent applications referenced in this application, the patents and patent applications referenced in those patents are also incorporated by reference in their entirety. In case of conflict, the present specification, including definitions, will control.

The details of one or more embodiments of the invention are set forth in the accompanying drawings and the description below. Other features, objects, and advantages of the invention will be apparent from the following drawings and detailed description.

### BRIEF DESCRIPTION OF THE DRAWINGS

FIG. 1 is a schematic illustrating an embodiment of a device for detecting the causative organism of a UTI that has multiple display windows and can be positioned in-line in a urinary catheter system.

FIG. 2 is a schematic illustrating another embodiment of a device for detecting the causative organism of a UTI that has multiple display windows and can be positioned in-line in a urinary catheter system.

FIG. 3 is a schematic illustrating an embodiment of a device for detecting the causative organism of a UTI that also tests for certain markers associated with organisms that cause UTIs.

FIG. 4 is a schematic illustrating an embodiment of a device for detecting the causative organism of a UTI that displays the name of the organism causing the UTI.

FIG. 5 is a schematic illustrating an embodiment of a device for detecting the causative organism of a UTI that displays the name of the organism causing the UTI.

FIG. 6 is a schematic illustrating a representative example of a UMD that may be adapted to accommodate the devices of the present inventions.

FIG. 7 is a schematic illustrating an embodiment of a device for detecting the causative organism of a UTI that is to be used in conjunction with a urinary catheter monitoring device.

FIG. 8 is a schematic illustrating an embodiment of a device for detecting the causative organism of a UTI that is to be used in conjunction with a urinary catheter monitoring device.

FIG. 9 is a schematic illustrating an embodiment of a device for detecting the causative organism of a UTI that is to be used in conjunction with a urinary catheter monitoring device.

Like reference symbols in the various drawings indicate like elements.

### DETAILED DESCRIPTION OF A PREFERRED EMBODIMENT

Nomenclature

| | |
|---|---|
| 100 | Device |
| 110 | Entry Port |
| 120 | Windows |
| 130 | Exit Port |
| 200 | Device |
| 210 | Sample Well |
| 220 | Detection Windows |
| 230 | Exit Port |
| 300 | Device |
| 310 | Sample Well |
| 311 | Entry Port |
| 320 | Result Windows |
| 322 | Result Window |
| 323 | Result Window |
| 324 | Result Window |
| 326 | Result Window |
| 330 | Exit Port |
| 340 | Box |
| 350 | Box |
| 400 | Device |
| 410 | Sample Wells |
| 411 | Entry Port |
| 420 | Detection Window |
| 422 | Detection Window |
| 430 | Exit Port |
| 600 | UMD |
| 610 | Entry Port |
| 620 | Result Window |
| 630 | Exit Port |
| 740 | Clip |
| 745 | Pierce Point |
| 746 | Pierce Point |
| 750 | Fastener |
| 755 | Fastener |
| 800 | Device |
| 810 | Piercing Member |
| 820 | Result Windows |
| 900 | Device |
| 910 | Piercing Member |
| 911 | Piercing Member |

Current methods for identifying the causative organism of a UTI are disparate, labor intensive, and time consuming It may be chemical or biochemical. Gram staining of the urine can give a preliminary indication of organism type, but not a specific identification. Clinically, gram staining is not routinely used. More commonly, a quantitative urine culture is performed in a clinical laboratory. This allows specific identification of the causative organism, and allows for testing to determine the antibiotic susceptibility and resistance characteristics of the organism, but the culture and sensitivity information may take forty-eight to ninety-six hours from submission of the sample to report of the culture results. Reducing the time and cost of receiving obtaining such information is desirable—and may be essential for certain vulnerable patients.

Detecting and treating UTIs often rely on an index of suspicion. The index of suspicion relies on observable signs and symptoms exhibited by the patient that might indicate an infection. Common signs noted by medical professionals include, but are not limited to, physical changes in the properties of the urine, such as discoloration of the urine, or blood in the urine (hematuria). Symptoms noted by the patient or clinician may also include urinary frequency (polyuria), burning with urination (dysuria), urgency, and suprapubic tenderness may also be noted. As an infection progresses, it may spread to the kidneys (pyelonephritis) and blood (sepsis). Additional signs and symptoms may include back and flank pain, fever, nausea, vomiting, leukocytosis, and other indicators of systemic infection.

Hopefully, early in the process of the infection, suspicion of the infection's presence will prompt further testing, evaluation, or treatment. Conventional methods of testing may be used in the non-catheterized patient. Once a positive screening test or confirmatory tests have been performed, therapy may be initiated. Based upon host factors, determination of causative organism may or may not be desirable. For example, in acute uncomplicated cystitis in healthy young women, urine culture or other modes of identifying the suspected pathogen may have little clinical utility. In more debilitated patients, such as those who have complicating factors such as an indwelling catheter or those with a more advanced infection such as pyelonephritis or signs of sepsis, determining the causative organism may help to direct therapy and improve outcomes.

UTIs are most often caused by bacteria, but UTIs can be caused by viruses, fungi, or protozoa. Typical causative organisms of UTI are Escherichia, Staphylococcus, Klebsiella, Enterococcus, Proteus, Morganella, Pseudomonas, and Candida species, though other organisms may be implicated. More generally, organisms responsible for UTI include bacteria such as Escherichia coli, Proteus mirabilis, Klebsiella, Pseudomonas aeruginosa, Serratia, Enterobacter, Staphylococcus epidermis, Enterococcus, Salmonella typhi, Mycobacterium tuberculosis, Staphylococcus aureus; viruses such as Polyomavirus JC, Polyomavirus BK, Cytomegalovirus, Adenovirus; fungi such as Candida albicans and other Candida spp., and Histoplasma capsulatum, and protozoa such Trichomonas vaginalis, Schistosoma haematobium. Thus, the DNA of these organisms, the antigens produced by these organisms, and the responses that these organisms trigger in the patient's body may be the markers that should be identified to diagnose and later treat the organism causing a UTI.

This patent discloses systems and devices for detecting multiple or a plurality of markers that are indicative of a UTI in urine. The number of organisms screened at a time may be 2, 3, 4, 5, 6, 7, 8, 9, 10, 11, 12, 15, 20, 25, etc.—the preferred number being a collection of organisms that in all likelihood represent the most likely candidates of causative organisms for a particular setting be it a patient, hospital, city, region, country, etc. The systems and devices provide sensitive, specific, and timely information regarding the causative organism of a UTI, which is of particular value to patients who have a urinary catheter system, e.g., an indwelling catheter. The inventions in this patent are especially useful for use in conjunction with the inventions of the UMD inventions.

The UMD inventions are urinary monitoring devices and methods that may provide early indication of the existence of a UTI in catheterized patients. Urinary catheter systems are known in the art, and generally include a urinary catheter, a collection tube, and a collection bag. Urinary catheter systems are made in a variety of sizes and from a variety of materials (e.g., latex or silicone) and include, for example, Foley catheters and straight catheters. See, for example, U.S. Pat. Nos. 4,575,371; 4,579,554; 4,813,935; 4,936,837; 5,295,979; 5,300,051; 5,785,694; 5,919,170; 6,117,120; 6,162,201; and 6,837,868 for descriptions of representative urinary catheter systems. By following the teachings of the UMD inventions, known catheters can be converted into urinary monitoring devices that provide an early indication of a UTI. A positive result using one or more of the UMD devices provides an indication that may prompt the use of the inventions of this patent. Another reason to use urine as an indicator and organism source is that urine provides a source of the causative organism, and the organism population may be concentrated in the urine sample. Following, or at times independent of, such early detection of a UTI, it may be advantageous to rule in or rule out the causative organism. Early detection of a UTI may save time, money, and lives.

When indicators of infection—such as a positive urinary monitoring device screening result, or the signs and symptoms noted above are present—the urine may be tested to identify a specific causative organism from among the various potential or probable causative organisms as described in this patent. A standing order as illustrated below may be used to expedite and simplify this process.

Depending on the test method used to identify the causative organism, the test method may use whole urine (which would likely include whole cells), urine isolates, urine that has undergone lysis steps to burst intact cells, whole cells isolated from urine, cellular fragments, or subcellular fragments that retain an enzymatic activity of interest, or antigen of interest. The testing may utilize known biology-based technologies such as protein-based or nucleic-acid-based technologies.

### Detection Technologies

Protein-based detection technologies capable of identifying a causative organism or markers associated with such organisms are known in the art and may be adapted for use in the present inventions. Specific antigen testing is widely accepted and practiced in the field of medicine. Examples of identification methods in clinical usage include rapid strep testing (group A strep), RSV, Legionella, pneumococcus, and influenza.

Useful methods include those known for identifying antigens used in Enzyme-Linked ImmunoSorbent Assay (ELISA) and other antibody-derived diagnostic tests. Monoclonal and polyclonal antibodies may be used. Methods for generating antigens and developing antibodies to the antigens are known. In fact, numerous textbooks describe such methods and there are companies in the business of generating such products for a fee. For example, books entitled The ELISA Guidebook (Methods in Molecular Biology) by John R. Crowther, The Immunoassay Handbook, Third Edition by David Wild, ELISA: Theory and Practice (Methods in Molecular Biology) by John R. Crowther are available.

ELISA kits are also available commercially, and the ELISA methods used in those kits may be adapted to the methods and devices of the present invention. Such kits include the hCG Combo test sold by Cardinal Health, QuickVue H. pylori gII test (and QuickVue+ Strep A test) sold by Quidel Corporation, BD Directigen Flu A+B (and Directigen RSV) sold by Becton, Dickson and Company, VIDAS Lyme IgG and IgM (and Vidas D-Dimer New (DD2)) sold by bioMerieux, Inc., and ImmunoCard Stat! Cryptosporidium/Giardia Rapid Assay sold by Meridian Bioscience, Inc.

Technology for implementing such devices is known. For example, home-use devices for the analysis of urine, for example in pregnancy tests and ovulation prediction tests, are available commercially, e.g., ClearBlue® easy and the E.P.T.® pregnancy tests. Patents disclosing this technology include U.S. Pat. Nos. 5,141,850, 5,366,863, 5,602,040, 5,622,871, 5,656,503, 6,187,598, 6,319,676, 6,352,862, 6,534,320, 6,767,714, and 7,045,342. Similarly, the devices of the present inventions may use the principles of immunochromatography, and include a hollow casing constructed of plastics material containing a porous assay strip carrying pre-dosed reagents. The reagents within the device may include one or more reagents labeled with a direct label, such as a dye sol, a metallic (e.g. gold) sol, or a colored latex (e.g. polystyrene) microparticle, which are visible to the eye when concentrated in a comparatively small test area of the strip. The user merely needs to apply a urine sample to one part of the casing to initiate the assay. As envisioned, a preferred embodiment of such a device would be configured to attach to a catheter or accept a urine sample collected from a catheterized patient suspected of having a UTI or CAUTI. In these types of devices, the assay result becomes visible by eye within perhaps a few minutes without further action by the user.

Sample collection can be conveniently achieved by placing the assay device in contact with a urine stream by means of a bibulous material that forms part of the device and that can readily take up sample from the urine stream. Optionally the bibulous material can protrude from the casing of the device to facilitate sample application. In addition to the specific examples of detectable materials already mentioned above, other materials can be used that block or reflect the electromagnetic radiation, rather than absorb it, e.g. “white” particles such as latex particles in their natural uncolored state. Alternatively, the label can be a reactant or catalyst that participates in the generation of a radiation absorbing or radiation-blocking material, e.g. an enzyme that reacts with a substrate to produce a detectable material, such as a colored material, in the detection zone.

The test strip can consist of any useful material. An optically diffuse layer of nitrocellulose or the like, perhaps sandwiched between two layers of optically clear film, e.g. of polyester such as Mylar can suffice. The clear film may protect the nitrocellulose where the assay reactions take place.

The constituent parts of the casing or housing can be molded from any materials, but it is contemplated that high-impact or similar plastics or polymers such as polystyrene and polycarbonate held together by push-fit clips or threaded screws or any other appropriate mechanism will work. It is generally envisioned that the material of the casing or housing will be opaque, e.g. white or colored plastics material, but the casing can be translucent or indeed transparent if desired. If the device must undergo sterilization care should be taken to choose materials that will withstand the sterilization procedure. Likewise, embodiments that are exposed to urine or other body fluids or chemicals should be selected to ensure that the materials can withstand the local environment to which it is exposed.

It will be appreciated that the overall layout and general shape of the monitor can be subject to very considerable variation from that described above without departing from the scope of the invention. The general shape and layout of the reading head is dictated by the need to cooperate effectively with the assay device but this shape can be varied considerably.

In some embodiments, the generation of a detectable color may signal a result. A result, of course can be positive (causative organism detected), negative (causative organism not detected) or a control (positive or negative). The result displayed by the device can be arranged to provide a “+” or “−” sign or to spell out words such as positive or perhaps even the name of the organism detected. The point being that any indication to the user will suffice, but signals that provide the user with further information may be preferred.

Methods for preparing porous or other materials to serve as the carrier and in the detection zones are known. For illustrative purposes only, a sheet porous material, e.g. nitrocellulose may cut into a plurality of identical assay strips. Parallel lines of assay reagents may be placed on the sheet before cutting. Reagents, e.g., a first immobilized antibody and perhaps a second different immobilized antibody are deposited on the carrier. Reagent deposition can be by means, including a pen or the like operated on a computer-controlled x-y plotting mechanism and fed with appropriate buffered reagent solution. If the sheet is nitrocellulose, reagents such as antibodies and antigens can be immobilized by simple direct application onto the nitrocellulose, followed by blocking of the sheet material, for example with albumen or polyvinyl alcohol.

Following reagent deposition and blocking, two lines of a mobile labeled reagent, such as a corresponding antigen or another antibody labeled for example with a particulate direct label such as colored latex, can be deposited. This deposition can be for example by means of another pen. Alternatively, the labeled reagents can be held in a separate porous pad or the like, rather than being applied directly to the test strip material.

Following all necessary reagent depositions and other treatments of the sheet, the sheet can be subdivided by cutting into individual strips. The exact position of the labeled reagent relative to the hole is not necessarily as critical as the location of the reaction zones, which should be arranged and sized to juxtapose the result windows.

By way of example only, the detection zone can be only a relatively small proportion of the total area of the strip. If appropriate for the purposes of the assay, multiple detection zones containing the same or different reagents can be placed on each strip. This may necessitate more than one labeled component being used; multiple mobile labeled components can be placed upstream on the strip or elsewhere within the device.

Methods for developing antibody-based diagnostics that may be adapted to the present inventions for bacteria, viruses, fungi, and protozoa are available.

For example, U.S. Pat. No. 6,841,154 discloses cross-reactive monoclonal and polyclonal antibodies that recognize surface proteins from coagulase-negative staphylococci and Staphylococcus aureus. U.S. Pat. No. 6,340,571 discloses antibodies specific for Staphylococcus aureus, including monoclonal or polyclonal antibodies specific for an epitope common to Staphylococcus aureus strains of various capsular serotypes, particularly methicillin-resistant strains, the antibody being selected from immunoglobulins G, M, and A, and the use thereof in a reagent for detecting Staphylococcus aureus. U.S. Pat. No. 6,194,161 discloses Staphylococcus aureus antigens and antibodies to the antigen are useful in kits and assays for diagnosing S. aureus infection.

U.S. Pat. No. 6,756,361 discloses Enterococcus antigens from strains of E. faecalis and E. faecium that can elicit production of protective antibodies. The antigens and antibodies to the antigens and methods disclosed may be useful in diagnostic assays. U.S. Pat. No. 6,617,156 discloses nucleic acid and amino acid sequences relating to Enterococcus faecalis for diagnostics and therapeutics.

U.S. Pat. No. 6,605,709 Nucleic acid and amino acid sequences relating to Proteus mirabilis for diagnostics and therapeutics.

United States Patents 7,029,684 and 6,551,795 disclose antigenic compositions of a Pseudomonas aeruginosa that may be used in diagnostic applications. U.S. Pat. No. 6,300,102 discloses immunogenic hybrid protein OprF-Oprl derived from Pseudomonas aeruginosa membrane proteins as well as to monoclonal or polyclonal antibodies against this hybrid protein. U.S. Pat. No. 5,716,829 discloses a diagnostic test for Pseudomonas aeruginosa infections.

U.S. Pat. No. 7,138,502 discloses antibodies to the propeptide of Candida albicans and methods of use. U.S. Pat. No. 6,916,626 discloses detection of Candida and a method of diagnosing Candida infections. U.S. Pat. No. 6,875,855 discloses nucleic acid and amino acid sequences of hemoglobin-response genes in Candida albicans and the use of reagents derived from these sequences in the diagnosis of disseminated Candida albicans infection. U.S. Pat. No. 6,287,833 a method for detecting Candida infection. U.S. Pat. No. 6,017,699 discloses PCR identification and quantification of important Candida species. U.S. Pat. No. 5,426,026 discloses PCR identification of four medically important Candida species using one primer pair and four species-specific probes that can detect and confirm four species of Candida, which allows for diagnosis of Candida infections. U.S. Pat. No. 4,806,465 discloses cytoplasmic antigens of Candida albicans and methods of using the same to generate polyclonal or monoclonal antibodies. U.S. Pat. No. 4,670,382 discloses monoclonal antibody to Candida albicans cytoplasmic antigens and methods of preparing same.

U.S. Pat. No. 4,803,156 discloses peptide-beta-lactamase conjugates for enzyme-linked immunoassays.

U.S. Pat. No. 7,029,684 discloses antigenic composition of a Pseudomonas aeruginosa and its use in diagnosing P. aeruginosa infections. U.S. Pat. No. 6,905,841 discloses enzymatic substrates for detecting Pseudomonas aeruginas. U.S. Pat. No. 6,893,817 discloses amino acid sequences and methods for detecting pseudomonas.

U.S. Pat. No. 6,605,709 discloses nucleic acid and amino acid sequences relating to Proteus mirabilis for diagnostics. U.S. Pat. No. 5,683,876 discloses nucleic acid probes and methods for detecting Proteus mirabilis.

Methods are also known for developing and using nucleotide primers to be used to identify organisms using PCR. For example, the LIGHTCYCLER-SEPTIFAST test sold by Roche Diagnostics GmbH can detect bacteria and fungi in whole blood samples. Methods like PCR may be used to genotype the organism at the same time as the organism is identified. This may assist in prescribing medicine to treat the UTI by identifying strains known to be resistant to certain antibiotics and the like. PCR methods are known and published in numerous books, including Rapid Cycle Real-Time PCR-Methods and Applications by Carl Wittwer, Meinhard Hahn, and Karen Kaul; PCR Protocols (Methods in Molecular Biology) by John M. S. Bartlett and David Stirling; PCR Protocols: A Guide to Methods and Applications by Michael A. Innis, David H. Gelfand, John J. Sninsky, and Thomas J. White; PCR Protocols: Current Methods and Applications (Methods in Molecular Biology) by Bruce A. White; and RT-PCR Protocols (Methods in Molecular Biology) by Joe Oconnell. Various patents on PCR methods for DNA or RNA also provide useful background material for PCR. See, e.g., U.S. Pat. Nos. 7,141,372, 7,101,663, 6,645,758, 6,492,114, 6,232,079, 6,180,349, 6,140,613, 5,229,297, 7,118,867, 6,881,559, 6,664,064, 5,618,703, 5,614,388, and 5,491,225.

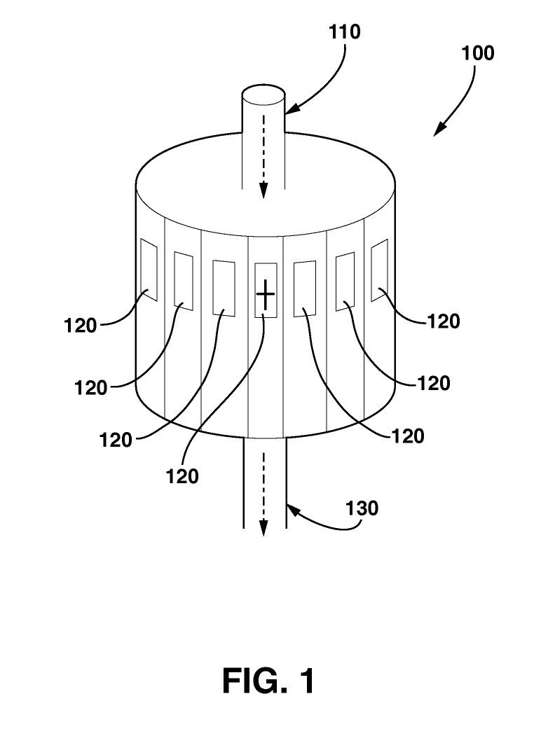

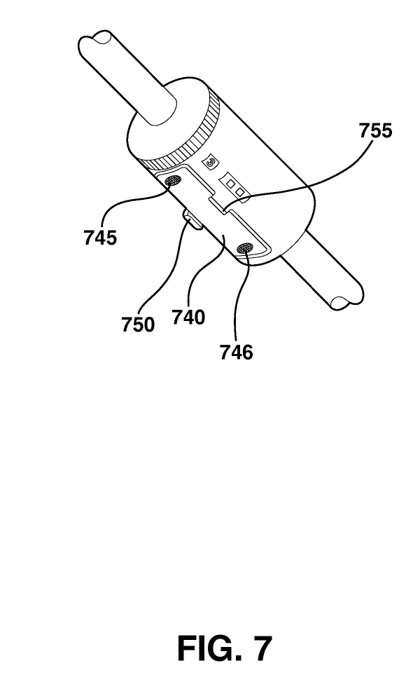

It should be understood that while FIGS. 1-9 depict embodiments that utilize ELISA technology, similar set ups may work for PCR-based applications as well. IN particular, the snap-in or piercing embodiments shown in FIGS. 7-9 can be modified to have a plurality of sampling wells or absorbent material that will capture samples to be then transferred to PCR sample wells. The captured samples can be spaced for easy transfer to the PCR wells. For example, if the PCR machine is set up to accompany 96-well PCR runs, the samples can be spaced and sized to transfer a proper amount of urine sample into each well. In this way, the chance of mishandling samples can be minimized. The sample capture piece can be part of the UMD or prepared separately and applied to the UMD only after a UTI is detected.

Dot blots may also be used as described in PCR Protocols: A Guide to Methods and Applications by Michael A. Innis, David H. Gelfand, John J. Sninsky, and Thomas J. White (see especially page 267), Protein Phosphorylation, Part A: Protein Kinases: Assays, Purification, Antibodies, Functional Analysis, Cloning, and Expression, Volume 200: Volume 200: . . . Part A (Methods in Enzymology) by John N. Abelson, Melvin I. Simon, Tony Hunter, and Bartholomew M. Sefton, and PCR Cloning Protocols (Methods in Molecular Biology) by Bing-Yuan Chen and Harry W. Janes. See also U.S. Pat. No. 5,059,522.

Once a preliminary identification has been made, specific treatment may be directed. Beyond identification of the causative organism, it may be useful to narrow in on the prevalent organisms in the region where the UTI is diagnosed. This may be useful because the probable causative organism may vary from region to region. It thus may be necessary to modify the methods disclosed herein to target specifically the most likely causative organisms according to the geographic region. See, e.g., Ann. Clin. Microbiol. Antimicrob., March 23; 6(1):4 (2007).

In addition, the treatment may be arrived on using global and local antibiotic susceptibility patterns. Thus, it may be useful to further type the causative organism. There are, for example, methicillin-resistant Staphylococcus Aureus (MRSA) strains. MRSA is resistant to certain antibiotics. These antibiotics include methicillin and other more common antibiotics such as oxacillin, penicillin and amoxicillin. Staph infections, including MRSA, occur most frequently among persons in hospitals and healthcare facilities (such as nursing homes and dialysis centers) who have weakened immune systems. MRSA infections that are acquired by persons who have not been recently (within the past year) hospitalized or had a medical procedure (such as dialysis, surgery, catheters) are known as community acquired MRSA infections. Staph or MRSA infections in the community are usually manifested as skin infections, such as pimples and boils, and occur in otherwise healthy people. For people with weakened immune systems, however, the consequences of an MRSA are more likely to be more severe and perhaps fatal. Thus, pursuing the diagnosis of MRSA infections is often warranted. There are methods known to detect MRSA, including those offered by Oxoid Limited, which offers a range of products for the detection of MRSA.

Enterococcus facelis and Morganella morganii are causative organisms that may warrant further classification. These gram-negative rod bacteria are known to become vancomycin resistant or VRE, which stands for vancomycin-resistant enterococci. Again there are methods for detecting VRE, including the ADVANDX VRE EVIGENE, a commercial vanA/vanB DNA hybridization assay. Other common methods including indole, citrate, and ornithine tests can detect VRE.

In addition to testing for the antigens that imply causality, antigens that imply other factors associated with the infecting organism may be tested for and detected. Key amongst these traits are those that confer antibiotic resistance, usually via antibiotic inactivation, alteration of the target site, or decreased access to the target site of antibiotic action. For example, certain bacteria excrete an enzyme b-lactamase, an enzyme that inactivates b-lactam antibiotics. Am mentioned above, resistance to vancomycin is most commonly due to production of a protein that prevents the antibiotic from binding to the cell wall. Other bacteria have altered cell wall channels that do not allow antibiotics to traverse the cell membrane, rendering those that act inside the cell ineffective. Common to all these mechanisms of resistance is the assertion that if the particular factors conferring bacterial resistance can be identified, they can likely be tested for and identified via antigen testing. In certain applications, PCR-based technology may be used to identify other factors associated with the infecting organism as well.

Detection devices may be may be manufactured to include a removable elements that may be utilized to facilitate the described bacterial-antigen testing. For example, an adsorbent filter paper, rayon pad, or other means to collect and/or concentrate antigens might be placed in contact with the urinary flow through the device, and subsequently removed at the time the device was scored with a positive indicator of infection. This removable element might then be processed in a fashion to analyze any adherent antigen in order to determine the causative organism. This arrangement is especially useful for PCR applications where the PCR instrument may be located in a hospital laboratory or on a different hospital or nursing home floor. In the non-catheterized patient, a representative urine sample may be spun down via centrifuge onto a similar pad or swab, which then may be processed in a similar fashion.

The Figures illustrate various embodiments of the inventions. It should be appreciated that these devices are applicable to any medical applications, including both human and veterinary applications. Further each of these designs may be mixed and matched with the various aspects depicted and can be designed to be stand-alone devices or adapted to be attached to a catheter.

FIG. 1 illustrates an ELISA-based device for detecting organisms that cause UTI. The device 100 may be constructed of any material but is likely to have a plastic housing that encases absorbent materials, also known as carriers that transfer an appropriate amount of a urine sample entering the device 100 at its urine sample entry port 110 to detection reservoirs that are juxtaposed result windows 120. The device 100 also contains an optional urine sample exit port 130. Entry port 110 and exit port 130 can be adapted to be attached directly to a catheter and thus receive, test, and expel urine as it exits the patient. The detection reservoirs are simply the area where the reaction takes place resulting in a visible indication if an organism of interest is detected. In device 100, the result window 120 in the center is displaying a “+” indicating a positive result. In this embodiment, each result window 120 is equipped to detect a different organism. Alternatively, detection reservoirs can be combined to detect more than one type of organism so long as the chemistry of detecting the various organisms are compatible. This may be useful to do when a group of organisms, if any one of which is detected, will result in the same treatment.

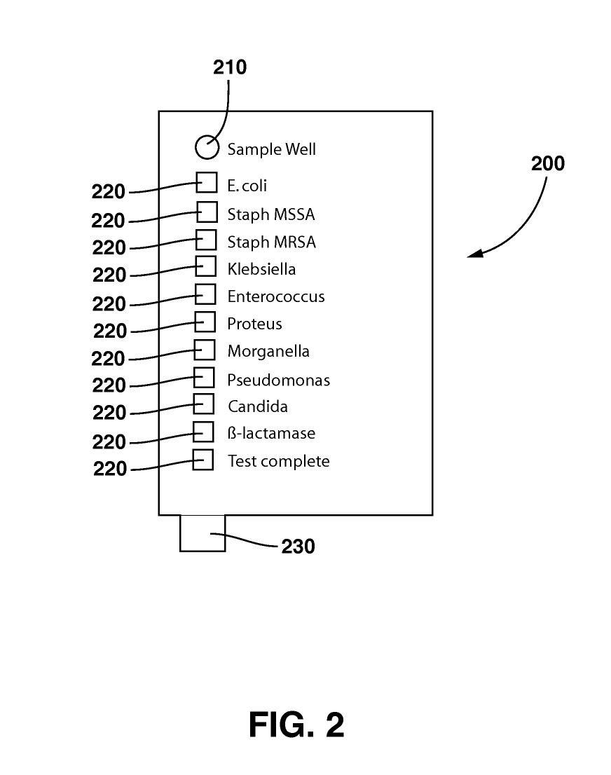

FIG. 2 illustrates a general schematic for a device 200. The sample well 210 can receive a sample directly from a catheter or from a urine sample previously collected. Carrier materials held within the casing of device 200 transfer the urine sample through the detection reservoirs that are detection windows 220. The detection windows 220 are labeled in this embodiment to indicate the organism each reservoir is adapted to test for. Sample well 210 can be adapted to connect to a catheter. The device 200 may contain an exit port 230.

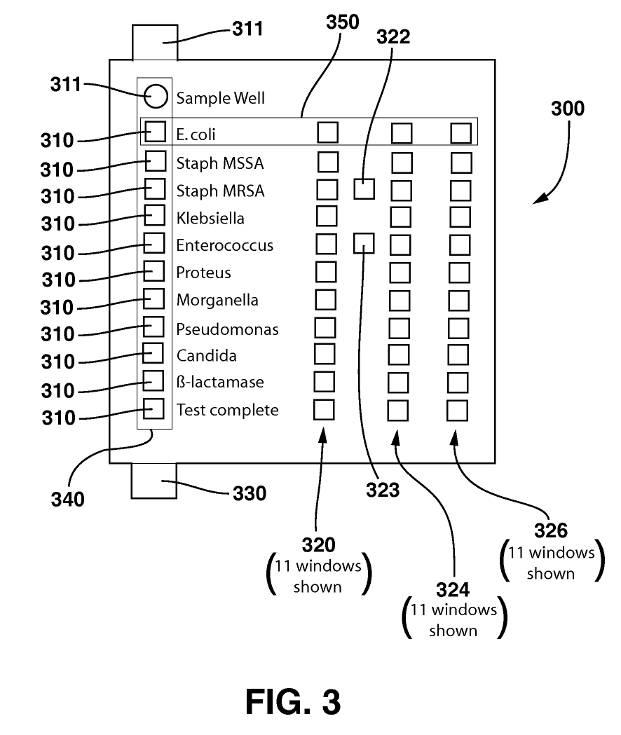

FIG. 3 illustrates a device 300 for detecting organisms that cause UTIs. A casing covers detection reservoirs made of absorbent carrier materials capable of eliciting a visual indication of an organism being present in a urine sample. Device 300 can have a plurality of urine sample wells 310 or entry port 311. Device 300 also has result windows 320, 322, 323, 324, and 326. Result windows 320 will display a positive result when a UTI-causing organism is detected. Result window 322 will display a positive result if a MRSA staph infection is detected. Result window 323 will display a positive result if a VRE resistant Enterococcus infection is detected. Result windows 324 are control windows indicating that the device 300 worked properly. Result windows 326 display a result when the test is complete. To accommodate the set up of device 300, carrier materials can be organized in device 300 to transfer urine aliquots vertically from an entry port 311 towards the exit port 330 and as indicated by box 340. Carrier materials may also be organized in device 300 to transfer urine aliquots horizontally to the detection reservoirs that are juxtaposed result windows result windows 320, 322, 323, 324, and 326. Such an arrangement is illustrated by box 350 for E. coli even though it is understood that separate carrier materials would be in place for each set of detection windows, i.e., each different organism to be tested.

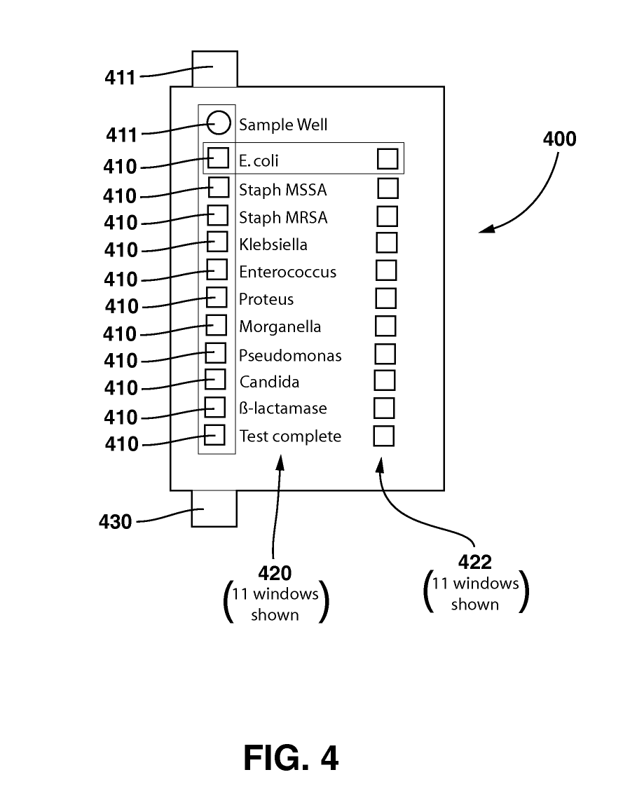

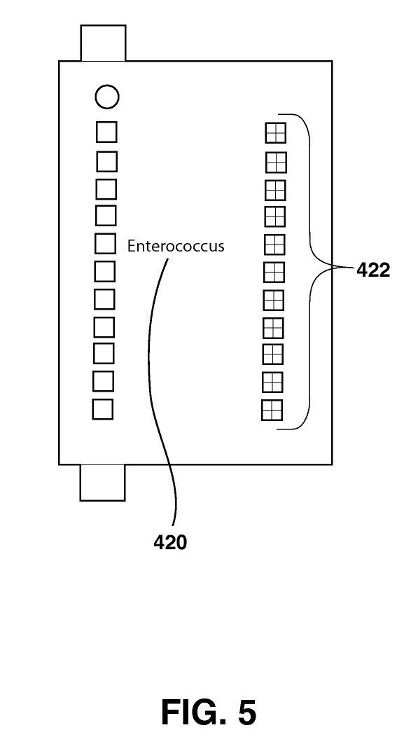

FIG. 4 illustrates a device 400 that is of similar design to device 300. It can have sample wells 410 or entry port 411, and exit port 430. Device 400 has detection windows 420 and 422. There may be multiple test-complete (control) detection windows 422. Detection windows 422 indicate that the test ran properly. Detection windows 420 display the name of the organism when the test is complete and each of the possible outcomes is shown in FIG. 4. In practice, detection of a single causative organism would likely display as shown in FIG. 5 where result windows 422 indicate the test ran properly for each set of test sets, and the causative organism is found to be Enterococcus.

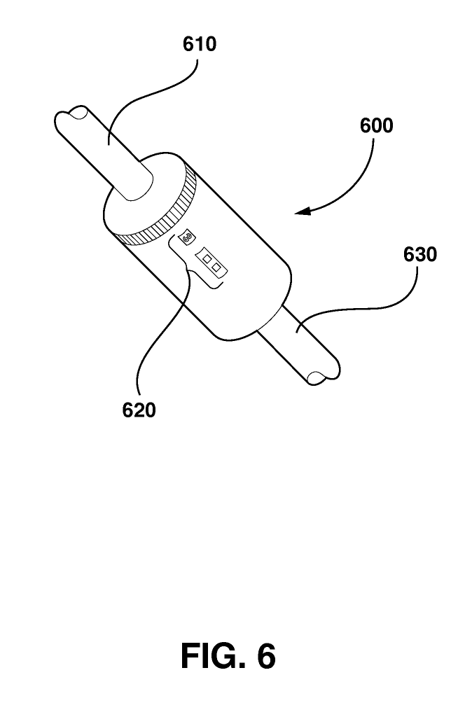

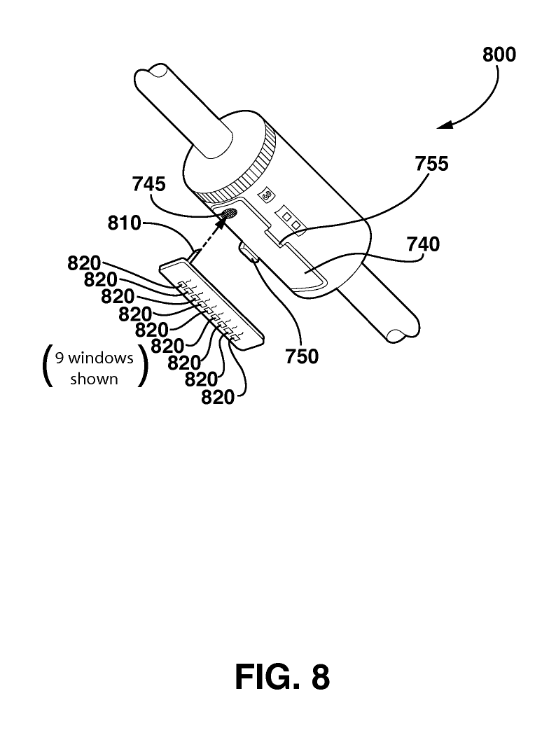

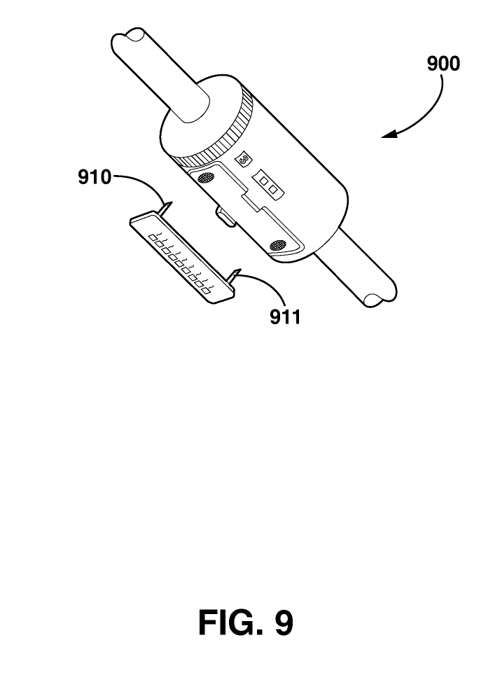

FIG. 6 illustrates a UMD 600 that may be adapted for use with the present inventions. UMD 600 has a urine entry port 610 and exit port 630. A result window 620 will indicate when a UTI is detected. FIG. 7 illustrates how the UMD 600 may be modified to receive a clip 740 with pierce points 745 or 746 (or both 745 and 746 as depicted in FIG. 7) and fasteners 750 and 755. FIG. 8 illustrates a device 800 with a single piercing member 810 that will fit in or pierce through pierce point 745. Device 800 is sized to snap into clip 740 and be held in place by fasteners 750 and 755. Piercing member 810 is the urine entry point for device 800. Urine proceeds into device 800 and a positive result for detecting a UTI-causing organism is visualized in result windows 820. FIG. 9 shows a modified version of device 800. Here device 900 contains two piercing members 910 and 911. In this way device 900 can have urine flow through the device 900 entering through piercing member 910 and exiting through piercing member 911.

The devices in FIGS. 1 through 9 can be modified to provide sample collection for subsequent PCR. To illustrate, the sample wells 310 can be modified to have a removable piece that samples urine. Preferably, the spacing of the wells 310 would coincide with the spacing of the sample wells used for the PCR testing. In this way, a sample for PCR can be removed from the device 300 and transferred for PCR testing. Alternatively, the devices in FIGS. 7-9 can be modified to contain a plurality of piercing members and pierce points to selectively remove samples for PCR testing. This can be done in conjunction with or separate from a design that tests for UTI-causing organisms with an antibody, chemical, or other design.

These designs are all adaptable for use with catheters. The basic designs can include a urine-sample receiver connected to a housing that encases at least one carrier, the carrier being configured to receive absorb or otherwise gather urine entering the housing from the urine-sample receiver. The carrier supplies the urine into a plurality of causative-organism-detection reservoirs. The reservoirs comprising the chemicals, biochemicals, or biologics described in this patent to detect the presence of a urinary-tract-infection organism. The reservoirs may be bounded by at least one result window located in the housing. The result window provides a visible indication of the presence or absence of the urinary-tract-infection organism in the urine.

A device for detecting the causative organism of a UTI can be positioned in-line in a urinary catheter system, such as a conventional Foley catheter. The Foley catheter systems often have a urinary catheter portion, a collection tubing portion, and a collection bag portion. A drainage outlet is also found on collection the bag. A housing can be configured to connect to an outlet or fitted with a separate attachment piece that allows the housing to receive urine from the Foley catheter system. The housing can be fitted to connect to the Foley catheter system at virtually any location along the urine path. It is preferable, however, to place it in line so that it does not increase the chances of introducing pathogens to the catheterized patient. It is also useful to locate it along the catheter pr tubing instead of coming out of the collection bag. The importance is to make sure the housing receives enough of a urine sample to allow a determination to be made. The amount of urine needed may vary according to the design of the housing and related detection materials.

In some embodiments, one or more portions of a urinary catheter system (e.g., a urinary catheter, a collection tube, or a collection bag) can contain a receptacle or the like into which a causative-organism device can be placed or into which urine can be collected and placed into contact with a causative-organism substrate. It is envisioned that receptacles or the like can be designed in a urinary catheter system that allow for removing one (e.g., a used) causative-organism device and replacing it with another (e.g., fresh) causative-organism device. Alternatively, receptacles or the like that allow an aliquot of urine to be separated from the remainder of the urine and placed into contact with a causative-organism device. Such a receptacle or the like should allow for the causative-organism device to contact the urine, however, it is understood by those in the art that the use of any type of releasable, removable, replaceable or attachable causative-organism device should not jeopardize the sterility and integrity of the closed urinary catheter system.

A causative-organism device can be placed on or in a urinary catheter system by a user (e.g., a nurse, attendant, or caretaker) or, as discussed above a user can bring a causative-organism device in contact with urine via a receptacle or the like. Alternatively, a causative-organism device can be manufactured as an integral part of a urinary catheter system.

The systems and devices described herein can result in a significant decrease in the morbidity and mortality of catheter-associated UTIs, which are usually very responsive to treatment once identified. Use of a system or device as disclosed herein also can result in substantial financial savings by cost-effectively treating UTIs at an early stage, thereby preventing the progression to more life threatening and costly systemic infections.

### Other Embodiments

It is to be understood that while the invention has been described in conjunction with the detailed description thereof, the foregoing description is intended to illustrate and not limit the scope of the invention. Other aspects, advantages, and modifications are within the scope of the invention.

1-31. (canceled)

32. A urinary catheter system for detecting the presence or absence of an organism that is indicative of a urinary tract infection (UTI) comprising
(a) a urinary catheter portion,
(b) a collection tubing portion, including at least
(1) multiple first UTI indicator substrates configured to be serially exposed to urine along the collection tubing portion, and capable of detecting and signaling the presence or absence of one or more markers in urine that are indicative of a UTI when exposed to urine, and
(2) a second UTI indicator substrate configured to be exposed to urine along the collection tubing portion, and capable of detecting the presence or absence of an organism in the urine, and
(c) a collection bag portion.

33. The system of claim 32 wherein the first UTI indicator substrates detect leukocyte esterase.

34. The system of claim 32 wherein the first UTI indicator substrates detect nitrites.

35. The system of claim 32 wherein the organism detected by the second UTI indicator substrate is selected from the group consisting of Escherichia, Staphylococcus, Klebsiella, Enterococcus, Proteus, Morganella, Pseudomonas, and Candida.

36. The system of claim 32 wherein the system further comprises (i) multiple second UTI indicator substrates retained within a first receptacle, wherein the second UTI indicators are configured to be exposed to urine along the collection tubing portion and capable of detecting the presence or absence of an organism in the urine, and (ii) a second receptacle configured to receive the first receptacle, wherein the second UTI indicator substrates are capable of receiving urine present in the system when the first receptacle is coupled to the second receptacle.

37. The system of claim 36 wherein the second UTI indicator substrates detect an organism selected from the group consisting of Escherichia, Staphylococcus, Klebsiella, Enterococcus, Proteus, Morganella, Pseudomonas, and Candida.

38. The system of claim 32 wherein the system further comprises a housing coupled to the second UTI indicator substrate, wherein the housing and second UTI indicator substrate are configured and arranged for selective nondestructive removal from the system.
